(19) United States
(12) Patent Application Publication
(10) Pub. No.: US 2018/0110504 A1
(43) Pub. Date: Apr. 26, 2018

(54) DISTRACTION TOOLS FOR SPINAL SURGERY

(71) Applicants: Paul C. McAFEE, Sparks, MD (US); Lukas EISERMANN, San Diego, CA (US)

(72) Inventors: Paul C. McAFEE, Sparks, MD (US); Lukas EISERMANN, San Diego, CA (US)

(21) Appl. No.: 15/794,589

(22) Filed: Oct. 26, 2017

Related U.S. Application Data
(60) Provisional application No. 62/413,186, filed on Oct. 26, 2016.

(51) Int. Cl. A61B 17/02; A61F 2/46; A61B 1/32; A61B 17/70; A61F 2/44

(52) U.S. Cl. CPC A61B 17/025; A61F 2/4611; A61B 1/32; A61B 34/20; A61F 2/4455; A61F 2/4657; A61B 2017/0256; A61B 17/7077

(57) ABSTRACT

Distraction tools that may be used in surgical operations, such as reconstructive spinal surgery, measuring spinal length and intervertebral spacing at the middle column, measuring intervertebral tension and establishing intervertebral spacer heights are disclosed. The distraction tools may be pneumatically operated and include distractor arms engagable with bone screws. Relative movement of the actuator arms adjusts the relative positions of the bone screws. In certain embodiments, the distraction tools are used to apply a desired amount of tension at the middle column of a spine utilizing air pressure to generate controlled distraction forces. The distraction tool systems may include touch-less gesture sensors, microcontrollers, and digital regulators.

### CROSS-REFERENCE TO RELATED APPLICATIONS

This application claims priority from U.S. Provisional Application No. 62/413,186 filed Oct. 26, 2016, which is incorporated herein by reference.

### FIELD OF THE INVENTION

The present invention relates to distraction tools for surgery, and more particularly relates to distraction tools for spinal reconstructive surgery that may be operated pneumatically.

### BACKGROUND INFORMATION

Various distraction tools have been used for spinal reconstructive surgery in which adjacent spinal vertebrae are manipulated into desired positions. However, a need exists for improved distraction tools for use in spinal surgeries and other types of surgery.

### SUMMARY OF THE INVENTION

The present invention provides distraction tools that may be used in surgical operations, such as reconstructive spinal surgery, measuring spinal length and intervertebral spacing at the middle column, measuring intervertebral tension and establishing intervertebral spacer heights. The distraction tools may be pneumatically operated and include distractor arms engagable with bone screws. Relative movement of the actuator arms adjusts the relative positions of the bone screws. In certain embodiments, the distraction tools are used to apply a desired amount of tension at the middle column of a spine utilizing air pressure to generate controlled distraction forces. The distraction tool systems may include touch-less gesture sensors, microcontrollers, and digital regulators.

An aspect of the present invention is to provide a distraction tool for spinal surgery comprising an actuator including a reciprocally movable piston mounted in a housing, a first distractor arm structured and arranged to releasably engage a first bone screw, a second distractor arm structured and arranged to releasably engage a second bone screw, and a linkage mechanism connected to the actuator piston and the first and second distractor arms, wherein the linkage mechanism is structured and arranged to move the first and second distractor arms in relation to each other upon the reciprocal movement of the actuator piston.

Another aspect of the present invention is to provide a pneumatic distraction tool system for spinal surgery comprising a pressurizable air cylinder, an actuator piston reciprocally movable in the pressurizable air cylinder, a first distractor arm structured and arranged to releasably engage a first bone screw, a second distractor arm structured and arranged to releasably engage a second bone screw, and a linkage mechanism connected to the actuator piston and the first and second distractor arms, wherein the linkage mechanism is structured and arranged to move the first and second distractor arms in relation to each other upon introduction of pressurized air into the pressurizable air cylinder, and further comprising a controller structured and arranged to control the level of air pressure introduced into the air cylinder.

These and other aspects of the present invention will be more apparent from the following description.

### BRIEF DESCRIPTION OF THE DRAWINGS

FIG. 1 schematically illustrates a pneumatic distraction tool system for spinal surgery in accordance with an embodiment of the present invention.

FIG. 2 is a partially schematic side view of a section of a spine illustrating the use of a distraction tool during a middle column procedure in accordance with an embodiment of the present invention.

FIG. 3 is a partially schematic side view of a section of spine illustrating the use of a distraction tool during a middle column procedure in accordance with an embodiment of the present invention.

FIG. 4 is a partially schematic front view of a portion of a spine illustrating the use of a distraction tool during a middle column procedure in accordance with an embodiment of the present invention.

FIG. 5 is a partially schematic side view of a section of spine including installed bone screws that may be manipulated with a pneumatic distraction tool in accordance with an embodiment of the present invention.

FIG. 6 is a partially schematic side view, and FIG. 7 is a partially schematic side sectional view, of a bone screw held by a distractor arm in accordance with an embodiment of the present invention.

FIG. 8 is an isometric view of a pneumatic distraction tool in accordance with an embodiment of the present invention.

FIG. 9 is a partially schematic side sectional view of a pneumatic distraction tool in accordance with an embodiment of the present invention.

FIG. 10 is a partially schematic side sectional view of The distraction tool of FIG. 9, with distractor arms thereof in an extended or distracted position.

FIG. 11 is a partially schematic side sectional view of a pneumatic distraction tool linkage mechanism in accordance with another embodiment of the present invention.

FIG. 12 is a schematic flow diagram illustrating operation of a pneumatic distraction tool in accordance with an embodiment of the present invention.

FIG. 13 is a schematic flow diagram illustrating operation of a pneumatic distraction tool in accordance with a further embodiment of the present invention.

### DETAILED DESCRIPTION

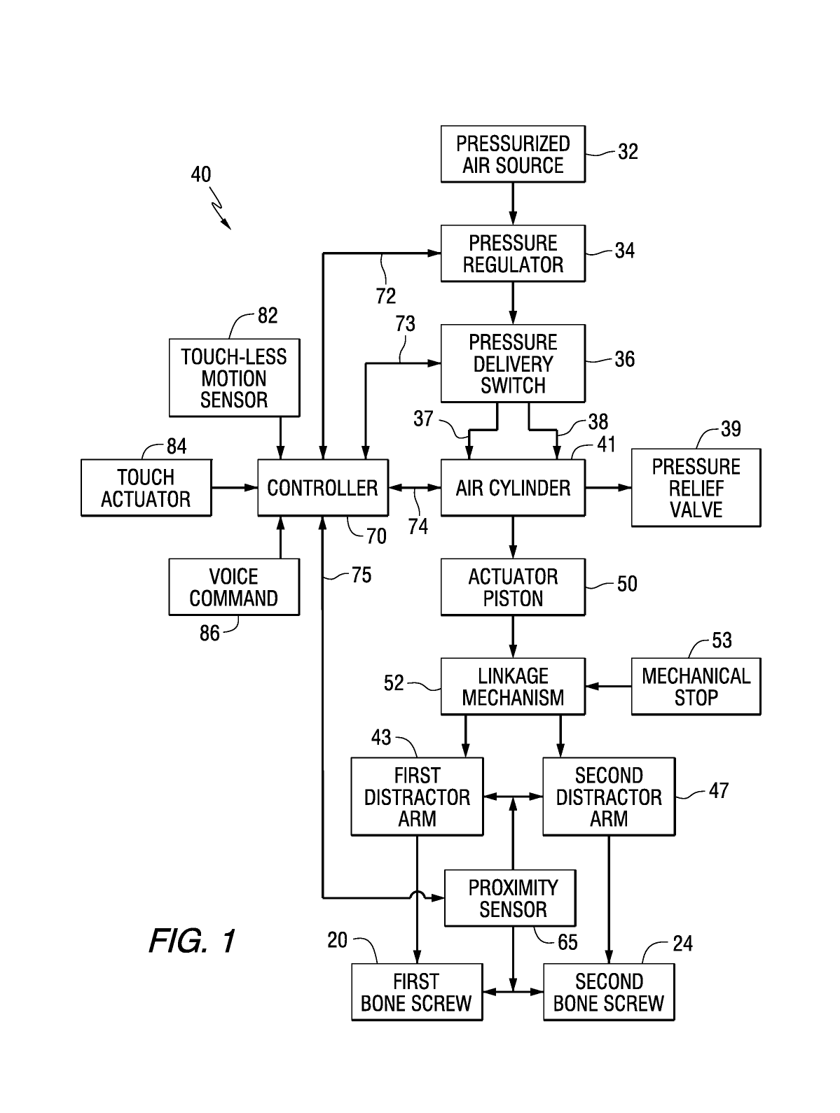

FIG. 1 schematically illustrates components and operation of a pneumatic distraction tool system 40 in accordance with an embodiment of the present invention. The pneumatic distraction tool 40 may be connected to first and second bone screws 20 and 24, shown at the bottom of FIG. 1 and described in further detail below. The pneumatic distraction tool 40 is connected to a pressurized air source 32 through a pressure regulator 34. In the embodiment shown, a pressure delivery switch 36 receives regulated air pressure from the pressure regulator 34, and selectively feeds the pressurized air through a first air supply line 37, or a second air supply airline 38, into an air cylinder 41. Although pneumatically actuated distraction tools are primarily described herein, it is to be understood that the distraction tools may be actuated by other means such as electromotors and the like.

As further shown in FIG. 1, an actuator piston 50 is reciprocally received inside the air cylinder 41, and is connected to a linkage mechanism 52. The linkage mechanism 52 is connected to a first distractor arm 43 and a second distractor arm 47. The first distractor arm 43 is releasingly engagable with the first bone screw 20, and the second distractor arm 47 is releasingly engagable with the second bone screw 24.

The air cylinder 41 may be equipped with a pressure relief valve 39, which may be used to maintain air pressure inside the air cylinder 41 at or below a maximum selected value to thereby limit the force applied to the actuator piston 50. A mechanical stop 53 may also be used to limit movement of the linkage mechanism 52 and/or the actuator piston 50 in order to limit the maximum travel distance between the first distractor arm 43 and second distractor arm 47. In the embodiment shown in FIG. 1, the mechanical stop 53 is provided at the linkage mechanism. Alternatively, the mechanical stop 53 may be provided at any other suitable location such as inside the air cylinder 41 in order to limit movement of the actuator piston 50.

As further shown in FIG. 1, a proximity sensor 65 may be used to determine the distance between the first distractor arm 43 and the second distractor arm 47 and/or to determine the distance between the first bone screw 20 and the second bone screw 24. As more fully described below, the proximity sensor 65 may comprise various known types of position sensors that are adapted for use during spinal distraction procedures in accordance with embodiments of the present invention to determine and limit the travel distances between the first and second bone screws 20 and 24 and/or between the first and second distractor arms 43 and 47.

As further shown in FIG. 1, a controller 70 is provided to control operation of the pneumatic distraction tool system 40. The controller 70 is connected by a signal line 72 to the pressure regulator 34, by a signal line 73 to the pressure delivery switch 36, by a signal line 74 to the air cylinder 41, and by a signal line 75 to the proximity sensor 65. Control inputs may be applied to the controller 70 by a touchless motion sensor 82, touch actuator 84 and/or voice command 86. As more fully described below, a surgeon or other operator may manipulate the controller 70 by selected tactile and/or non-tactile means during a surgical procedure, e.g., by hand motions.

Although a motion sensor may be used to actuate the distraction tool, it is to be understood that any other suitable type of touchless sensor may be used. Furthermore, actuation may be controlled using any suitable mechanical device such as a button, dial, toggle switch, joystick and the like that can be sterilized prior to the procedure. Furthermore, actuation may be achieved by voice commands, for example, using a smartphone via Bluetooth with a matching app. A microcontroller may be used in the actuation system, and may be connected in any conventional way such as Ethernet, wi-fi, Bluetooth, USB and the like.

In certain embodiments, the distractor tools of the present invention are used during spinal surgery procedures involving the middle column of the spine. As used herein, the term “middle column” means a region running along the Y-axis of the spine and extending along the Z-axis that is bounded on one side by the posterior surface of each vertebral body in an area near the posterior longitudinal ligaments (PLL), and is bounded on another side (measured along the Z-axis) by a distance substantially one-third of the distance through the vertebral body measured from the posterior surface of the vertebral body in the Z-axis, i.e., from the posterior side to the anterior side of each vertebral body. It is to be understood that the anterior boundary of the middle column is substantially at the one-third distance (33.3 percent), but the anterior boundary may extend up to 50 percent of the distance through the vertebral body measured long the Z-axis, i.e., the middle column may nominally range of from 0 percent to 33.3 percent, but may range up to 50 percent in certain embodiments.

In accordance with embodiments of the present invention, pneumatic tools are provided for distracting, tensioning and/or translating spinal segments. The system provides the ability to calculate and quantify the force, displacement and stiffness in the determination of the presence or absence of spinal instability. For example, in the lumbar spine, this may be distraction, translation, or side-to-side ligamentous laxity of 3 mm or more. There can be also excessive angular motion of greater than 11 degrees. The pneumatic distraction tools of the present invention may be used to detect excessive physiologic relationships between the two vertebral segments. As more fully described below, the distraction instrument may include a piston parallel to the middle column to measure distraction and compression and/or a piston perpendicular to the bone anchor may also be provided in order to measure translation or shear motion. The amount of air pressure applied to the piston may be used to control the amount of distraction. The distraction distance at the middle column may be measured, and correlated with the amount of force applied by the distraction tool.

Flow of pressurized air into the pneumatic cylinder 41 of the present distraction tools 40 may be controlled by a motion sensing device. For example, when an operator's hand is passed over the motion sensor, air is delivered at a controlled pressure to the pneumatic cylinder of the distraction tool, which causes the distractor arms to move away from each other to thereby separate the adjacent vertebrae along the Y-axis. The operator may pass his or her hand over the motion sensor in an opposite direction, causing a control signal to be sent to reduce the pressure delivered to the pneumatic cylinder, thereby reducing the distraction force and allowing the distractor arms and corresponding attached vertebrae to move closer together along the Y-axis.

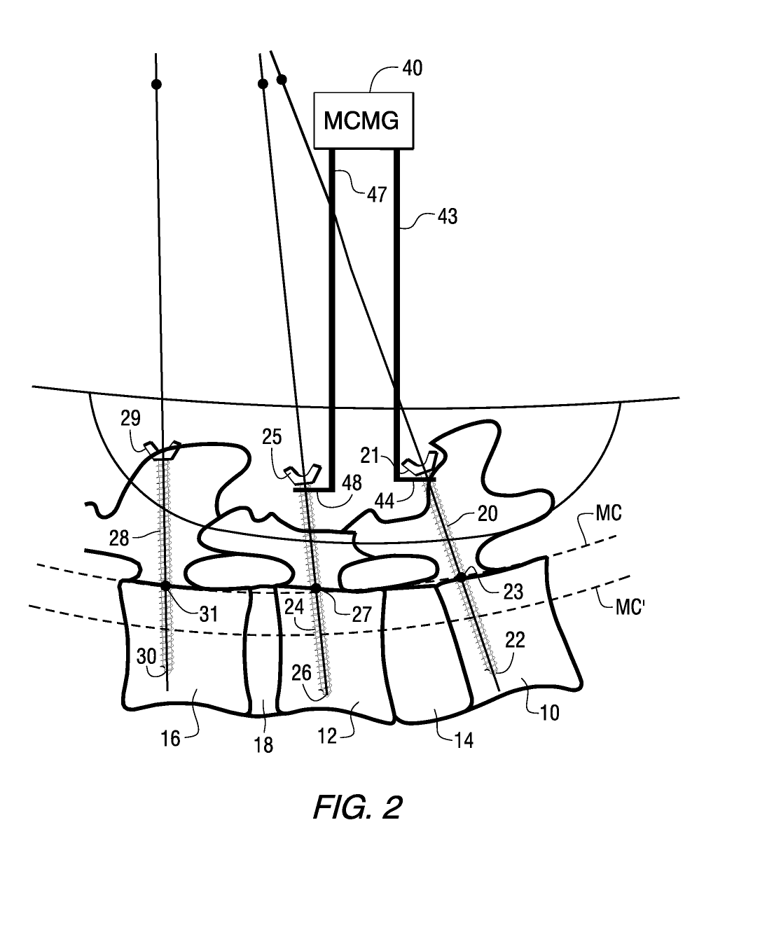

In accordance with embodiments of the invention, the distraction tool 40 may be used to perform various spine surgery procedures. FIG. 2 is a partially schematic side view of a section of a spine including three vertebrae 10, 12 and 16, each of which has a bone anchor in the form of a bone screw 20, 24, 28 installed therein. FIG. 2 shows the posterior middle column line MC, and the anterior middle column line MC′, which is located at a distance one-third of the diameter of each vertebra, measured from their posterior sides. The first bone screw 20 includes a head 21 and tip 22. The second bone screw 24 includes a head 25 and tip 26. The third bone screw 28 includes a head 29 and tip 30. As further shown in FIG. 2, each bone screws 20, 24 and 28 include a central column marker 23, 27 and 31 that is located on or near the posterior middle column line MC when the screw is installed in the vertebra. The middle column markers 23, 27 and 31 may comprise any suitable types of detectable features, such as a shoulder, recess, dissimilar material, or the like. FIG. 2 also schematically shows a distraction device 40 of the present invention including two distractor arms 43 and 47, each of which is connected near the heads 21 and 25 of the first and second bone screws 20 and 24.

The distraction tool 40 may be provided in the form of a middle column measurement guide or gauge (MCMG) that may be utilized in a posterior approach to the lumbar spine. Bone screws or posted bone screws may be used such that the surgeon or operator can use fluoroscopy and determine from the outer silhouette of the screw or other detectable feature exactly the depth of screw insertion to the middle osteoligamentous column where the posterior longitudinal ligament lies in the lateral projection. The screws can be placed in lordosis, kyphosis or alternate angles as long as the depth down to the middle column can be ascertained. In this manner, the stresses, axial height, and rotational position of the middle column can be determined. The user may directly measure the distance and the force of distraction and the forces of compression placed along the middle column. The middle column measurement guide allows surgeons to directly measure the force of correction and the tension of ligamentotaxis along the posterior longitudinal ligament.

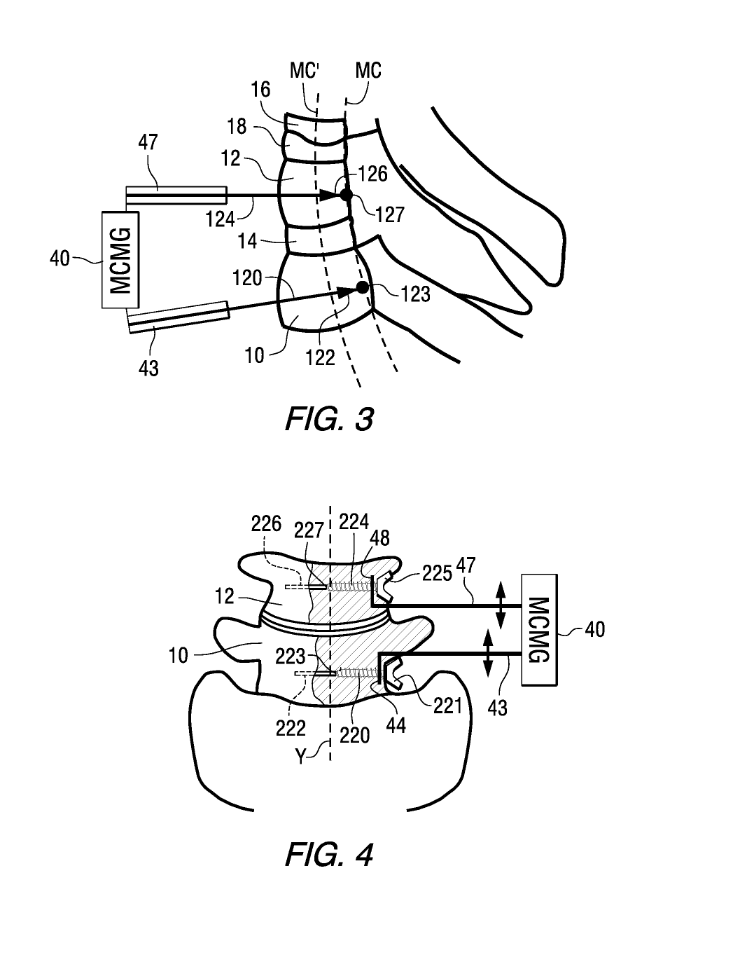

FIG. 3 is a partially schematic side view of a section of a spine in which middle column markers in the form of screws or pins have been installed from an anterior side of each vertebra. A first bone screw or pin 120 having a tip 122 is installed in the vertebra 10, and a second bone screw or pin 124 having a tip 26 is installed in the vertebra 12. The tip 122 of the pin 120 is located at or near a point 123 within the middle column, i.e., at the posterior middle column line MC. The tip 126 of the pin 124 is located at or near a point 127 within the middle column, i.e., at the posterior middle column line MC. A distraction device 40 including distractor arms 43 and 47 attached to the screws or pins 120 and 124 is also schematically shown in FIG. 3.

The embodiment shown in FIG. 3 may use the middle column measurement gauge through Caspar pins which are inserted from the anterior part of the cervical spine, e.g., during anterior cervical discectomy and fusion. The Caspar pins or any rod or anchoring screws placed in the anterior aspect of the cervical vertebral body may be inserted in various neutral or flexion-extension angles to the depth of the middle column. Although the Caspar pins can be placed in lordosis or kyphosis, the tip of each pin may be used as the measuring point lying within the middle column. This configuration of the triaxial quality of middle column measurement guide is advantageous because this makes the anchor points and Caspar pins not dependent on having a parallel or orthogonal orientation with regard to each other. The reference point of three calculated measurements (linear displacement; angular displacement or motion; and strain or stress) may be used to find effective displacements and moments from the tip of the cranial Caspar pin to the tip of the caudal Caspar pin to determine the displacements and moments along the middle column of the spine. The bone anchors can be temporary in order to assess the requirement for a fusion, or they can be permanent anchors intended to be incorporated directly into a fusion instrumentation construct, either minimally invasively, mini-open, or open surgery.

FIG. 4 is a partially schematic front view of a portion of a spine in which bone screws have been installed laterally into each vertebra. A first bone screw 220 is installed in a vertebra 10. The bone screw 220 includes a head 221, tip 222 and transition marker 223. A second bone screw is installed in another vertebra 12. The bone screw 224 includes a head 225, tip 226 and transition marker 227. As schematically shown in FIG. 4, the distraction tool 40 includes a first distractor arm 43 attached to the first bone screw 220 near its head 221, and a second distractor arm 47 connected to the second bone screw 224 near its head 225.

FIG. 4 illustrates a lateral thoracolumbar approach, for example a lateral lumbar interbody fusion LLIF, extreme lateral interbody fusion XLIF, or direct lateral interbody fusion DLIF. Dual diameter screws having shoulders are shown. Even though an operating table may be hinged, and the screws that are placed into the vertebral bodies may be placed at an angle, the middle column parameters can be determined. If a dual diameter screw is used, or a screw that has a marking on the outer silhouette which can be visualized fluroscopically, the depth from the middle column measurement guide down to the middle osteoligamentus column can be accurately determined. This may be used for a lateral lumbar interbody fusion LLIF (DLIF or XLIF) with supplemental fixation. The supplemental fixation may serve a dual purpose—it is also used for compression-distraction of the disc space which is being prepared for the use and implantation of an interbody spacer through a direct lateral approach.

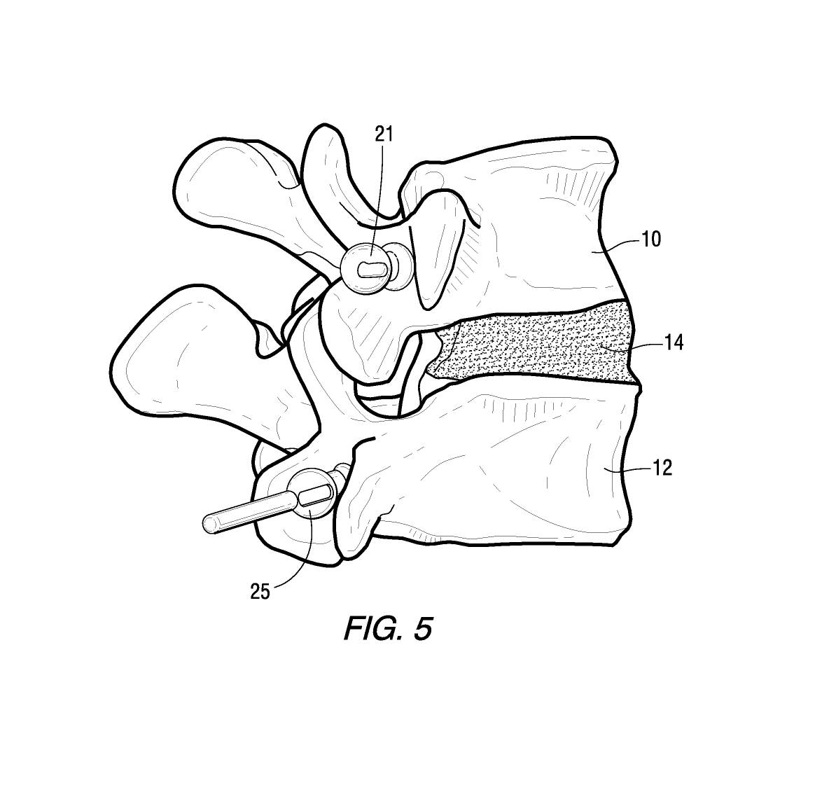

FIG. 5 illustrates a lumbar spine section 10, 12 and 14 that may undergo a posterior-approach similar to the embodiment shown in FIG. 2. The first and second bone screws shown in FIG. 5 include generally round heads 21 and 25 with engagable slots through, with one of the heads 25 having a pin extending therefrom.

In accordance with embodiments of the present invention, the distraction tools may be used to move and measure distances between adjacent vertebrae at the middle column along the Y-axis (middle column gap balancing) as described in U.S. application Ser. No. 15/344,320 to Paul McAfee entitled “Methods and Apparatus for Spinal Reconstructive Surgery, Measuring Spinal Length and Intervertebral Spacing at the Middle Column, Measuring Intervertebral Tension and Establishing Intervertebral Spacer Heights” filed Nov. 4, 2016, which is incorporated herein by reference.

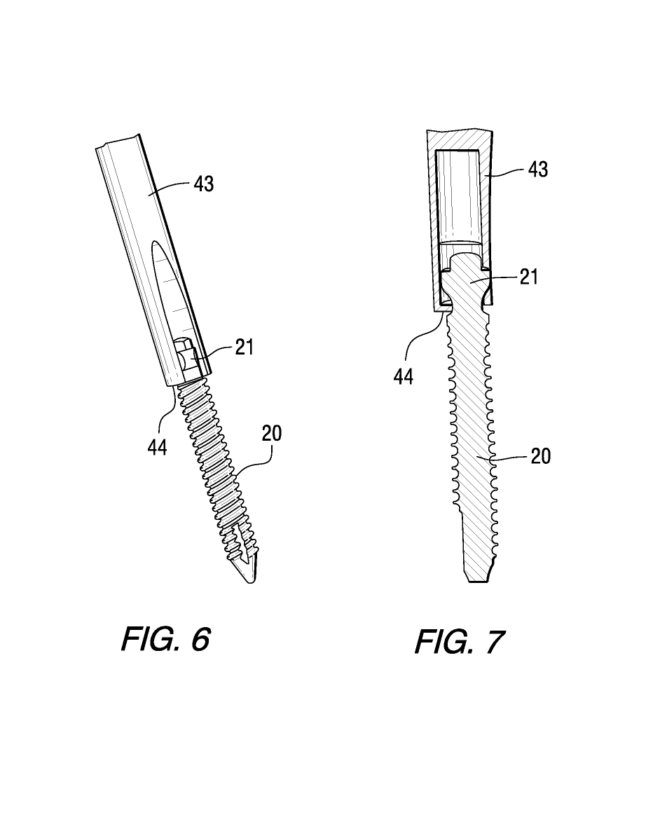

FIGS. 6 and 7 illustrate a portion of the distractor arm 43 of a distraction tool with an engagement tip 44 that releasingly engages the head 21 of the first bone screw 20. In the embodiment shown, the head 21 of the bone screw 20 is radially elongated in one direction and flattened in another direction to allow the head 21 to fit into a slot of the engagement tip 44. The head 21 may be retained in the slot of the engagement tip 44 by a 90° rotation of the distractor arm 43 around the axial direction of the bone screw 20, e.g., by a bayonette-type engagement, or by any other suitable type of releasable fastening structure known to those skilled in the art.

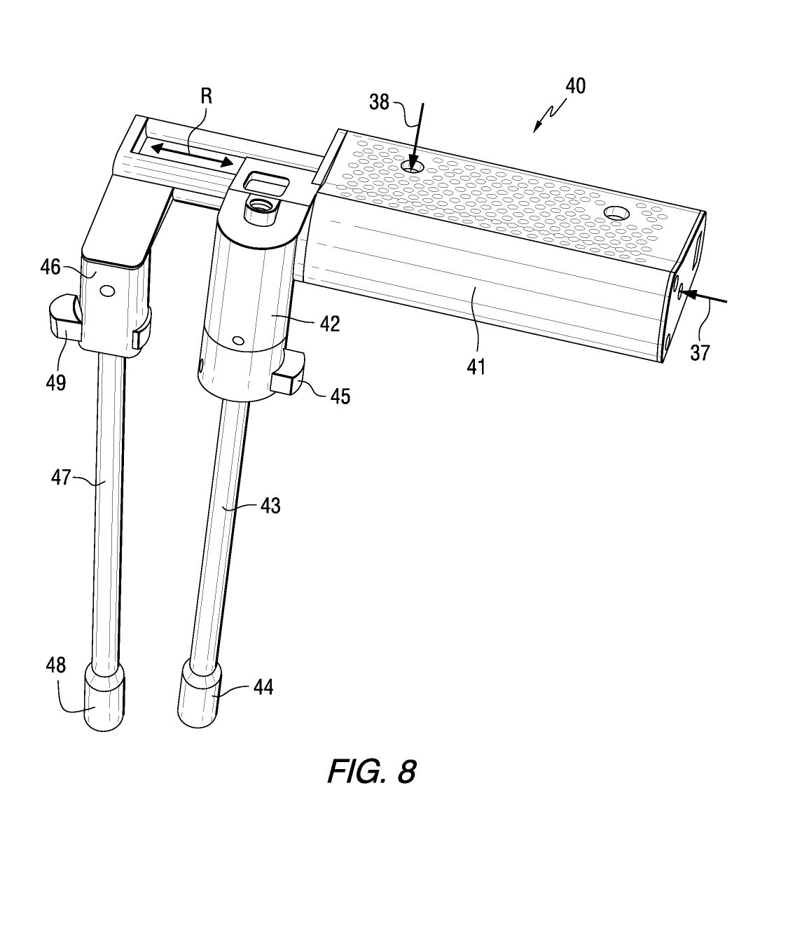

FIG. 8 is an isometric view of a pneumatic distraction tool 40 in accordance with an embodiment of the present invention. The distraction tool 40 includes a housing 41 having a pneumatic cylinder located therein. A first distractor arm mounting assembly 42 attached to the housing 41 has a first distractor arm 43 releasably mounted thereon. The distractor arm 43 includes an engagement tip 44 that can be releasably secured to or contact a bone screw or pin, as described above. A locking mechanism 45 may be used to releasably secure the distractor arm 43 in the distractor arm mounting assembly 42. A second distractor arm mounting assembly 46 is slidably mounted in relation to the housing 41, and is capable of reciprocating movement R with respect to the housing 41. A second distractor arm 47 is releasably mounted on the second arm mounting assembly 46. The second distractor arm 47 includes an engagement tip 48 that can be releasably secured to or contact another bone screw or pin, as described above. A locking mechanism 49 may be used to releasably secure the second distractor arm 47 in the second distractor arm mounting assembly 46. The second distractor arm assembly 46 is mounted on a piston that moves within the pneumatic cylinder in the housing 41. In accordance with embodiments of the invention, the distance of travel along the direction R may be manually or automatically measured in order to provide a measurement of relative movement between the first and second distractor arms 43 and 47. A source of pressurized air may be connected to the pneumatic cylinder 41 via a port 37. Delivery of air at controlled pressures causes the first and second distractor arms 43 and 47 to move in relation to each other.

The opposing first and second distractor arms 43 and 47 may engage with the first and second bone screws 20 and 24 attached to the adjacent spinal vertebrae 10 and 12 as shown in FIG. 2, as well as the screws and pins shown in FIGS. 3-5. The first and second distractor arms 43 and 47 are forced away from each other through the use of the pneumatic piston in the housing 41. The pneumatic piston forces the distractor arms 43 and 47 away from each other to thereby increase the spacing between the adjacent vertebrae 10 and 12, e.g., along the Y-axis of the spine. The amount of air pressure applied to the piston controls the amount of distraction. The distraction distance at the middle column may be measured, and correlated with the amount of force applied by the distraction tool 40.

The distraction tool 40 may thus include distractor arms that are lockably mounted on respective mounting bracket assemblies. Relative movement of the distractor arms may be controlled by a pneumatic cylinder contained within a handle that may be grasped and manipulated by the surgeon. The degree of relative movement between the distractor arms may be indicated by markings viewable to the surgeon, e.g., the markings may be used to indicate the spacings between the distractor arms at the beginning and end of a distraction procedure, and such measurements may be correlated with the amount of distraction force applied by the pneumatic cylinder during the procedure.

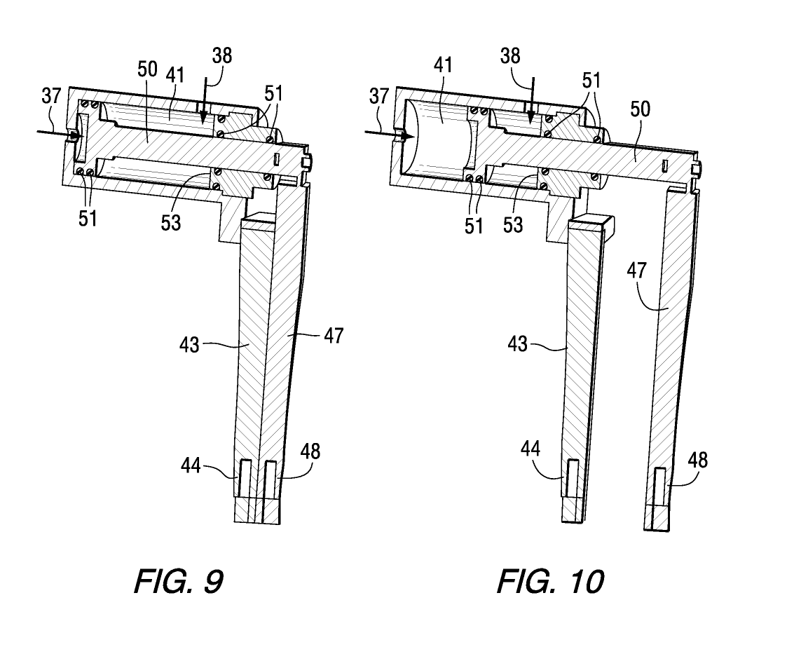

FIGS. 9 and 10 are partially schematic side sectional views illustrating components of a pneumatic distraction tool 40 in accordance with an embodiment of the present invention. The distraction tool 40 includes an air cylinder 41 having an actuator piston 50 reciprocally movable therein. The actuator piston 50 is sealed within the cylinder 41 by means of multiple o-ring seals 51. A linkage mechanism 52 controls relative movement of the first distractor arm 43 and the second distractor arm 47. In the embodiment shown, the linkage mechanism, includes a rigid attachment of the first distractor arm 43 to the housing of the pressure cylinder 41, and a rigid attachment of the second distractor arm 47 to the actuator piston 50. The first and second distractor arms 43 and 47 are movable from a fully retracted position shown in FIG. 9 to a distracted or extended position shown in FIG. 10.

As further shown in FIGS. 9 and 10, the first air supply line 37 feeds into the rear of the air cylinder 41, while the second air supply line 38 feeds into a front portion of the air cylinder 41. When pressurized air is fed through the first air supply line 37, the pressurized air forces the actuator piston 50 from the position shown in FIG. 9 to an extend position such as that shown in FIG. 10. Alternatively, when pressurized air is fed through the second air supply line 38, the actuator piston 50 may be forced from the extended position shown in FIG. 10 to the fully retracted position shown in FIG. 9. During typical reconstructive spine surgeries, distraction of adjacent spinal vertebrae away from each other is achieved by the force resulting from pressurized air that is introduced into the air cylinder 41 through the first supply line 37. However, in certain procedures, it may also be desirable to forcibly pull or retract adjacent spinal vertebrae toward each other, in which case it may be desirable to supply pressurized air through second supply line 38. Although the second supply line 38 is shown as entering through a side of the air cylinder housings in FIGS. 8-11, it is to be understood that the feed line may be located at any suitable location, e.g., the second supply line 38 may run from an end of the cylinder housing along its length to the desired inlet position into the air cylinder.

In accordance with embodiments of the invention, the distraction tool 40 is designed so that it can be autoclaved repeatedly, hence the choice of air as the means for functioning. An air tool needs only a piston and an air hose inside the sterile field to operate. The equipment to control the air pressure can be located outside the sterile field. This allows for a wide range of conventional electronics components to be utilized in its design. The components that will be repeatedly sterilized can be made from conventional metal or polymer materials that can survive repetitive autoclave cycles as well as repetitive EtO or Gamma radiation cycles.

The pressurized chamber is (or chambers are) located outside the patient's body. This is done to reduce the risk associated with potential mechanical or operator failures. It also allows the use of relatively large cross-section pistons, which in turn allows the use of low (and thus safe) pressures to achieve the desired mechanical forces.

A basic mechanical function of the distraction tool 40 is to apply force to the spine. This is achieved by using one or more pneumatic pistons. The pistons apply constant, predictable force based on the air pressure inside the chamber. This force can be calculated based on the geometry of the piston and/or it may be measured and calibrated based on measuring the force generated by the piston and plotted vs. the input pressure in the piston. The microcontroller can be programmed to correct for any sort of input-output curve correction that may be required to accommodate deviations from the expected linear conversion curve.

By utilizing various combinations of pistons, the spine can be manipulated in either isolated or complex motion planes. The current embodiments may utilize one piston to apply force in the axial direction of the spine along the Y-axis. This is the primary motion utilized for middle column balancing, and also the primary motion employed during surgery to distract the disc space. This piston may be referred to as the distraction piston.

An embodiment may also utilize a second piston, e.g., mounted in an orientation 90 degrees away from the axial piston. This second piston may have a dual-chamber design, permitting force to be applied in the direction of an anterior slip (spondylolisthesis) or in the direction of a posterior slip (retrolisthesis). Thus, distraction may be performed in the X-axis and/or Z-axis. This piston may be referred to as the listhesis piston. The second piston may be situated directly beneath a connector that allows the instrument to be attached to a table-mounted arm. The use of a table-mounted arm minimizes rotational displacements, since applying force with the second piston will apply a moment tending to cause rotation of the instrument about itself and about the attachment point. In addition to the use of a table-mounted arm to counteract rotational movement, any other suitable type of mechanical constraint may be used to minimize unwanted rotational displacement.

In accordance with certain embodiments, a proximity sensor 65 as illustrated in FIG. 1, or similar position sensors, may be used to detect relative positions of the first and second distractor arms 43 and 47, and/or detecting the relative positions of the first and second bone screws 20 and 24 (e.g., at the central column markers thereof 23 and 27), thereby making it possible to measure the amount of distraction achieved by the instrument. Examples of sensors known to those skilled in the art that may be adapted for use in accordance with the present invention include soft potentiometers, magnetic angle position sensors, proximity sensors, magnetic field sensors, Hall effect sensors, or other means of measuring distance. Feeding this information back to the microcontroller allows the creation of automated robotic control loops that can manipulate the spine in a number of useful ways.

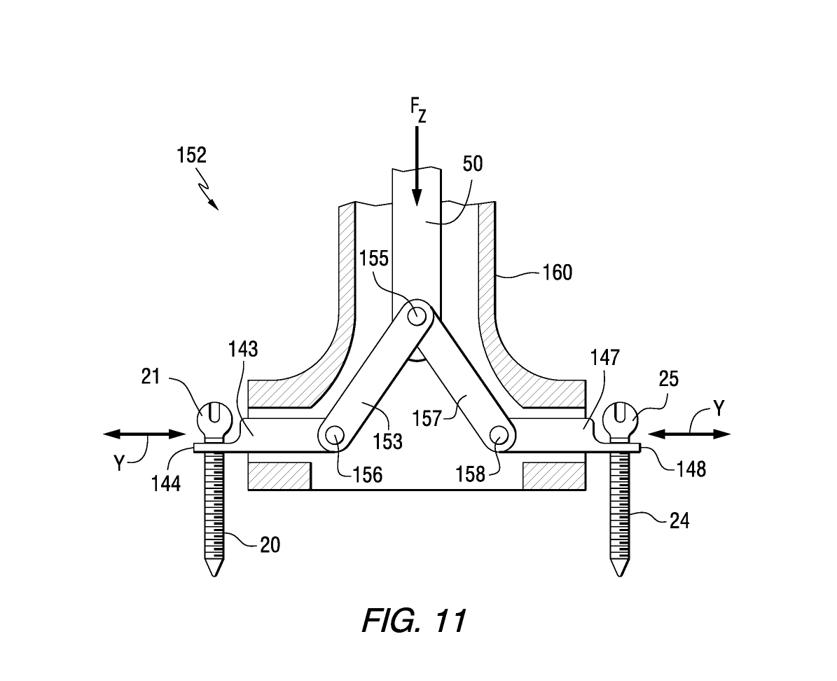

FIG. 11 is a partially schematic side sectional view illustrating a distraction tool linkage mechanism 152 in accordance with another embodiment of the present invention. In the embodiment shown in FIG. 11, the linkage mechanism 152 is actuated by the actuator piston 50, similar to that shown in the previous embodiments of FIGS. 8-10. However, as shown in FIG. 11, a first distractor arm 143 having an engagement tip 144 engages the head 21 of the first bone screw 20 at an orientation that is substantially perpendicular to the axial direction of the first bone screw 20. Similarly, the second distractor arm 147 includes an engagement tip 148 engaging the head 25 of the second bone screw 24. The orientation of the second distractor arm 147 is generally perpendicular to the axial direction of the second bone screw 24. A first linkage arm 153 connects the actuator piston 50 to the first distractor arm 143 by means of a pivot joint 155 at one end thereof and a pivot joint 156 at another end thereof. Similarly, the second linkage arm 157 is connected between the actuator piston 50 and second distractor arm 147 by means of the first pivot joint 155 and another pivot joint 158. In the embodiment shown, the first and second linkage arms 153 and 157, and the pivot connections 155-156, and 158, are contained within a housing 160 of the linkage mechanism 152.

In the configuration shown in FIG. 11, the actuator piston 50 may be reciprocally movable in a direction Fz, e.g., with respect to a piston cylinder 41 as described above. Reciprocal movement of the actuator piston 50 with respect to the air cylinder 41 along the direction of the arrow Fz shown in FIG. 11 causes the first and second actuator arms 143 and 147 to move toward and away from each other in a direction substantially perpendicular to the direction of travel of the actuator piston 50. As described above, the distance of travel along the direction Fz may be manually or automatically measured and, taking into account the linkage geometry, can be used to determine relative positions of the first and second distractor arms 143 and 147, and their respective first and second bone screws 20 and 24.

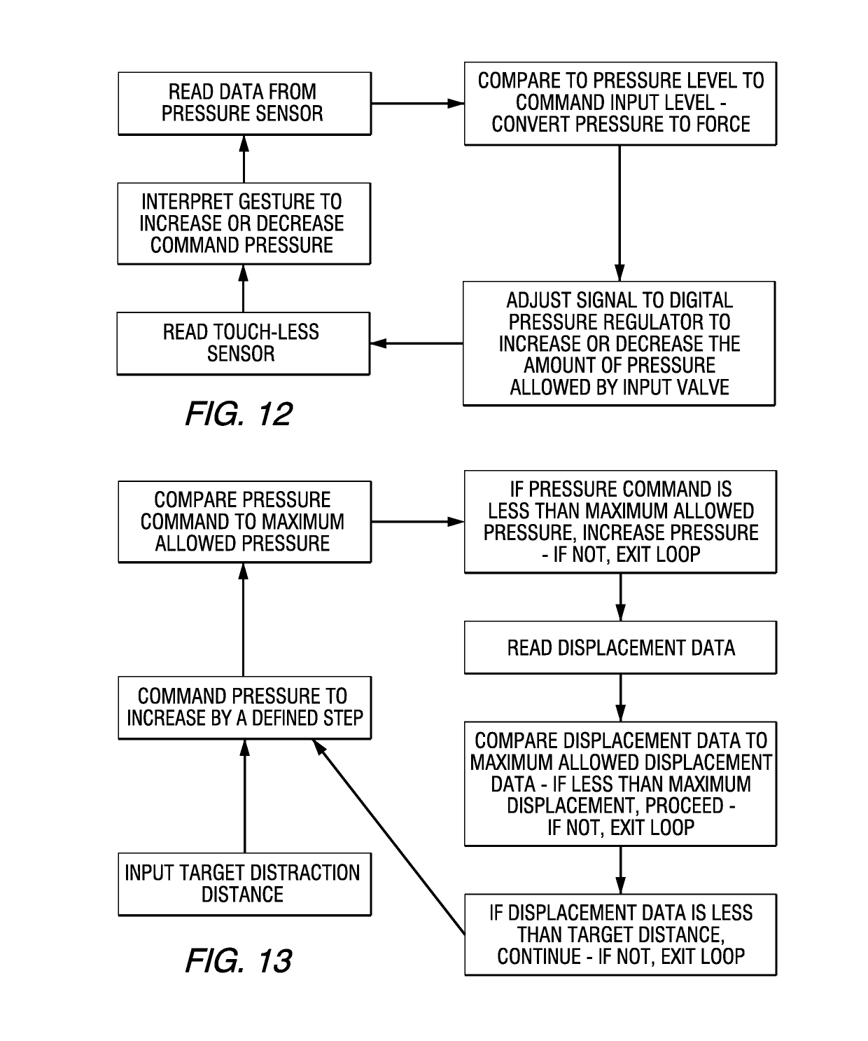

A basic control loop is schematically illustrated in FIG. 12, in which a surgeon or other operator may make a hand gesture which is read by a touchless sensor. The hand gesture is interpreted to increase or decrease command pressure, and a standard pressure sensor may be used to measure pressure and generate corresponding pressure data. The sensed pressure level may then be compared to a command level input, and the pressure level may be correlated with a corresponding force. An adjusted signal may then be sent to the pressure regulator to increase or decrease the amount of pressure introduced into the air cylinder, e.g., by an input valve.

The basic control loop illustrated in FIG. 12 may thus include the steps of: read data from pressure sensor; compare to pressure level to command input level; convert pressure to force; adjust signal to digital pressure regulator to increase or decrease the amount of pressured allowed by input valve; read a sensor such as a touchless motion sensor or mechanical control button; and interpret sensor information to increase or decrease command pressure.

A middle column gap balance control loop is schematically illustrated in FIG. 13, in which a target distraction distance is determined and inputted into the system, and a pressure command signal is used to increase pressure inside the air cylinder by a selected amount. The increased pressure is compared to a maximum allowed pressure, and if the increased pressure is less than a maximum allowed pressure, pressure is then further increased. If not, no increased pressure command is generated and the loop is exited. Displacement data is then read and compared to maximum allowed displacement data. If the measured displacement is less than a maximum displacement, additional pressure may be applied resulting in additional displacement. If the measured displacement data reaches the maximum allowed displacement, no further pressure is applied, no further displacement is produced and the loop is exited. If the displacement data is less than the target distance, the loop is continued.

The middle column gap balance control loop illustrated in FIG. 13 may thus include the steps of: input target distraction distance; command pressure to increase by a defined step; compare pressure command to maximum allowed pressure; if pressure command is less than maximum allowed pressure, increase pressure; if not, exit loop; read displacement data; compare displacement data to maximum allowed displacement data; if less than maximum displacement, proceed; if not, exit loop; if displacement data is less than target distance, continue; if not, exit loop.

The target distraction distance may be input as a numerical value by the surgeon, or it may be input via software means based on image analysis of the middle column distance. If the target distance is determined by image analysis, then that image analysis can be updated iteratively as new fluoroscopy images are made, allowing continually improving accuracy.

In accordance with embodiments described above, a basic mechanical function of the distraction tool 40 is to apply force to the spine, e.g., by using one or more pneumatic pistons. The piston(s) apply constant, predictable force based on the air pressure inside the chamber. This force can be calculated based on the geometry of the piston and/or it may be measured and calibrated based on measuring the force generated by the piston and plotted vs. the input pressure in the piston. A microcontroller can be programmed to correct for any sort of input-output curve correction that may be required to accommodate deviations from the expected linear conversion curve. By utilizing various combinations of pistons, the spine can be manipulated in either isolated or complex motion planes. An embodiment may utilize one piston to apply force in the axial direction of the spine along the Y-axis. This is the primary motion utilized for middle column balancing, and also the primary motion employed during surgery to distract the disc space.

The distraction tool 40 may be utilized to perform the middle column gap balancing procedure described herein. For example, based on a pre-operative fluoroscopy scan, the spinal length at the middle column is measured, and then the target axial distraction distance is calculated based on restoring the spine to its natural anatomic position, e.g., when the PLL is straightened and tensioned. Either by applying a known force and monitoring progress by proximity sensor(s) and/or fluoroscopy, or by providing the target distance to the microcontroller and allowing the distraction tool to apply force as needed, the target may be reached. To ensure that the procedure is performed safely, upper limits of force and distraction distance may be programmed into the control software. These limits may also be physically designed into the tool by means of pressure relief valves that actuate above a certain air pressure and/or mechanical stops to prevent excess motion. Both air pressure limits and mechanical stops may be provided as features adjustable by the surgeon.

The force and resulting motion achieved may be plotted on a force-displacement graph. This graph can be used to assess the degree of stability in the spine. For example, a current medical guideline suggests that a spinal motion segment which moves 3 mm or more on flexion-extension x-ray analysis should be fixated by spinal fusion, whereas a spinal motion segment moving less than this should not be fused. Distraction instruments can apply the force necessary to move the spine in an objective, controlled manner, while simultaneously recording the resultant motion.

Additionally, by attaching a communications means, such as a Bluetooth chip, an Ethernet card, or other means of exporting a digital signal, to the microcontroller, the instrument is capable of sending the information gathered to a storage device. The storage device may be any form of computer memory, memory attached to an electronic device such as a printer, or may be uploaded to a database on the internet. The information can then be utilized as part of an electronic record of the surgery. It may be a standalone record or may be combined with the outputs of other devices used during the surgery, such as the anesthetic record.

Used singly, distraction tools of the present invention may be utilized to perform the middle column gap balancing procedure. Based on the pre-operative fluoroscopy scan, the spinal length (e.g., the path along the PLL) is measured, and then the target axial distraction distance is calculated based on restoring the spine to its natural anatomic position (e.g., when the PLL is straightened and tensioned). Either by applying a known force and monitoring progress by fluoroscopy, or by providing the target distance to the microcontroller and allowing the distraction tool to apply force as needed, the target may be reached. To ensure that the procedure is performed safely, upper limits of force and distraction distance may be programmed into the control software. These limits may also be physically designed into the tool by means of pressure release valves that actuate above a certain air pressure and/or mechanical stops to prevent excess motion. Both air pressure limits and mechanical stops could be fixed in manufacturing, or could be provided as features adjustable by the surgeon.

The force and resulting motion achieved may be plotted on a force-displacement graph. This graph can be used to assess the degree of stability in the spine. For example, a current medical guideline suggests that a spinal motion segment which moves 3 mm or more on flexion-extension x-ray analysis ought to be fixated by spinal fusion, whereas a spinal motion segment moving less than this ought not to be fused. The instrument of the current embodiment can apply the force necessary to move the spine in an objective, controlled manner, while simultaneously recording the resultant motion.

Additionally, by attaching a communications means—such as a Bluetooth chip, an Ethernet card, or other means of exporting a digital signal—to the microcontroller, the instrument is capable of sending the information gathered to a storage device. The storage device may be any form of computer memory, memory attached to an electronic device such as a printer, or may be uploaded to a database on the internet. The information can then be utilized as part of an electronic record of the surgery. It may be a standalone record or may be combined with the outputs of other devices used during the surgery, such as the anesthetic record.

It is not required to limit the use of the distraction tools to a single device. An array of distraction tools may be utilized to create complex patterns of force application. Such an array could be utilized to precisely correct spinal deformities. For example, a bone anchor (such as one described in U.S. Pat. No. 8,974,507) could be placed into the bones of each vertebra that is intended to be surgically manipulated. An instrument could be placed across each pair of bone anchors, and then under the surgeon's touch-free control, each instrument could individually—or in combination—apply a known, precise, controlled force to effect surgical correction of a spinal deformity. This allows many more points of force application than a surgeon, who has only two hands at his disposal, could manage with currently known tools. It also allows much more precise (and thus safe) load application than is possible with the human hand. Finally, unlike a human hand, the instrument will not fatigue. Thus, force can be maintained on individual spinal segments throughout lengthy spinal procedures. The surgeon will also be able to gauge the degree of spinal stability at each operative level and make intraoperative decisions based on this information.

In certain embodiments, the instrument is not limited only to the traditional operative setting. It is possible to insert the bone anchors and attach the instrument under local anesthesia in a minimally invasive manner. By doing so, the surgeon may be able to diagnose spinal stability through direct force application in an office setting with or without the use of x-ray. Additionally, by keeping the patient awake, simple tests of pain sources could be carried out. For example, by applying distraction force to a degenerative spine segment, feedback about whether or not pain relief is achieved may be obtained from the patient in real time. Since the test is computer-controlled, placebo cycles can easily be programmed to verify whether pain relief is physical or psychological.

The distraction tools of the present invention can be table mounted or freehand. Pressure sensors and lights can be utilized as indicators on a programmable control board to indicate the cycling or stepwise addition of progressive force application. The instrument can be used as a spinal tensioning, distracting or translating device that is anchored directly to the vertebra at adjacent levels for the purpose of determining spinal ligamentous laxity in determining the indication for spinal instrumentation and/or bony fusion procedure.

Any element expressed herein as a means for performing a specified function is intended to encompass any way of performing that function including, for example, a combination of elements that performs that function. Furthermore, the invention, as may be defined by such means-plus-function claims, resides in the fact that the functionalities provided by the various recited means are combined and brought together in a manner as defined by the appended claims. Therefore, any means that can provide such functionalities may be considered equivalents to the means shown herein.

In various embodiments, various models or platforms can be used to practice certain aspects of the invention. For example, software-as-a-service (SaaS) models or application service provider (ASP) models may be employed as software application delivery models to communicate software applications to clients or other users. Such software applications can be downloaded through an Internet connection, for example, and operated either independently (e.g., downloaded to a laptop or desktop computer system) or through a third-party service provider (e.g., accessed through a third-party web site). In addition, cloud computing techniques may be employed in connection with various embodiments of the invention.

Moreover, the processes associated with the present embodiments may be executed by programmable equipment, such as computers. Software or other sets of instructions that may be employed to cause programmable equipment to execute the processes may be stored in any storage device, such as a computer system (non-volatile) memory. Furthermore, some of the processes may be programmed when the computer system is manufactured or via a computer-readable memory storage medium.

It can also be appreciated that certain process aspects described herein may be performed using instructions stored on a computer-readable memory medium or media that direct a computer or computer system to perform process steps. A computer-readable medium may include, for example, memory devices such as diskettes, compact discs of both read-only and read/write varieties, optical disk drives, and hard disk drives. A computer-readable medium may also include memory storage that may be physical, virtual, permanent, temporary, semi-permanent and/or semi-temporary. Memory and/or storage components may be implemented using any computer-readable media capable of storing data such as volatile or non-volatile memory, removable or non-removable memory, erasable or non-erasable memory, writeable or re-writeable memory, and so forth.

A “computer,” “computer system,” “computing apparatus,” “component,” or “computer processor” may be, for example and without limitation, a processor, microcomputer, minicomputer, server, mainframe, laptop, personal data assistant (PDA), wireless e-mail device, smart phone, mobile phone, electronic tablet, cellular phone, pager, fax machine, scanner, or any other programmable device or computer apparatus configured to transmit, process, and/or receive data. Computer systems and computer-based devices disclosed herein may include memory and/or storage components for storing certain software applications used in obtaining, processing, and communicating information. It can be appreciated that such memory may be internal or external with respect to operation of the disclosed embodiments. In various embodiments, a “host,” “engine,” “loader,” “filter,” “platform,” or “component” may include various computers or computer systems, or may include a reasonable combination of software, firmware, and/or hardware. In certain embodiments, a “module” may include software, firmware, hardware, or any reasonable combination thereof.

In general, it will be apparent to one of ordinary skill in the art that various embodiments described herein, or components or parts thereof, may be implemented in many different embodiments of software, firmware, and/or hardware, or modules thereof. The software code or specialized control hardware used to implement some of the present embodiments is not limiting of the present invention. Programming languages for computer software and other computer-implemented instructions may be translated into machine language by a compiler or an assembler before execution and/or may be translated directly at run time by an interpreter. Such software may be stored on any type of suitable computer-readable medium or media such as, for example, a magnetic or optical storage medium. Thus, the operation and behavior of the embodiments are described without specific reference to the actual software code or specialized hardware components. The absence of such specific references is feasible because it is clearly understood that artisans of ordinary skill would be able to design software and control hardware to implement the embodiments of the present invention based on the description herein with only a reasonable effort and without undue experimentation.

Various embodiments of the systems and methods described herein may employ one or more electronic computer networks to promote communication among different components, transfer data, or to share resources and information. Such computer networks can be classified according to the hardware and software technology that is used to interconnect the devices in the network, such as optical fiber, Ethernet, wireless LAN, HomePNA, power line communication or G.hn.

The computer network may be characterized based on functional relationships among the elements or components of the network, such as active networking, client-server, or peer-to-peer functional architecture. The computer network may be classified according to network topology, such as bus network, star network, ring network, mesh network, star-bus network, or hierarchical topology network, for example. The computer network may also be classified based on the method employed for data communication, such as digital and analog networks.

As employed herein, an application server may be a server that hosts an API to expose business logic and business processes for use by other applications. The application servers may mainly serve web-based applications, while other servers can perform as session initiation protocol servers, for instance, or work with telephony networks.

Although some embodiments may be illustrated and described as comprising functional components, software, engines, and/or modules performing various operations, it can be appreciated that such components or modules may be implemented by one or more hardware components, software components, and/or combination thereof.

The flow charts and methods described herein show the functionality and operation of various implementations. If embodied in software, each block, step, or action may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processing component in a computer system. If embodied in hardware, each block may represent a circuit or a number of interconnected circuits to implement the specified logical function(s).

As used herein, “including,” “containing” and like terms are understood in the context of this application to be synonymous with “comprising” and are therefore open-ended and do not exclude the presence of additional undescribed or unrecited elements, materials, phases or method steps. As used herein, “consisting of” is understood in the context of this application to exclude the presence of any unspecified element, material, phase or method step. As used herein, “consisting essentially of” is understood in the context of this application to include the specified elements, materials, phases, or method steps, where applicable, and to also include any unspecified elements, materials, phases, or method steps that do not materially affect the basic or novel characteristics of the invention.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. In this application and the appended claims, the articles “a,” “an,” and “the” include plural referents unless expressly and unequivocally limited to one referent.

Whereas particular embodiments of this invention have been described above for purposes of illustration, it will be evident to those skilled in the art that numerous variations of the details of the present invention may be made without departing from the invention as defined in the appended claims.

What is claimed is:

1. A distraction tool for spinal surgery comprising:
an actuator comprising a reciprocally movable piston mounted in a housing;
a first distractor arm structured and arranged to releasably engage a first bone screw;
a second distractor arm structured and arranged to releasably engage a second bone screw; and
a linkage mechanism connected to the actuator piston and the first and second distractor arms, wherein the linkage mechanism is structured and arranged to move the first and second distractor arms in relation to each other upon the reciprocal movement of the actuator piston.

2. The distraction tool of claim 1, wherein the actuator piston is reciprocally movable in a pressurizable air cylinder upon introduction of pressurized air into the pressurizable air cylinder.

3. The distraction tool of claim 2, wherein the linkage mechanism releasably secures the first distractor arm to the air cylinder, and releasably secures the second distractor arm to the actuator piston.

4. The distraction tool of claim 2, wherein the linkage mechanism includes a first linkage arm pivotally connected to an end thereof to the actuator piston and pivotally connected to another end thereof to the first distractor arm, and a second linkage arm pivotally connected at an end thereof to the actuator piston and pivotally connected at another end thereof to the second distractor arm.

5. The distraction tool of claim 2, further comprising a proximity sensor structured and arranged to detect variable distances between the first and second distractor arms.

6. The distraction tool of claim 2, further comprising a pressure sensor structured and arranged to detect variable pressures applied between the first and second distractor arms.

7. The distraction tool of claim 2, further comprising means for controlling air pressure introduced into the air cylinder to thereby control relative movement between the first and second distractor arms or control force applied between the first and second distractor arms.

8. The distraction tool of claim 2, further comprising a controller structured and arranged to control the level of air pressure introduced into the air cylinder.

9. The distraction tool of claim 8, wherein the controller receives inputs from at least one of a motion sensor, touch actuator and voice command to thereby control the level of air pressure.

10. A pneumatic distraction tool system for spinal surgery comprising:
a pressurizable air cylinder;
an actuator piston reciprocally movable in the pressurizable air cylinder;
a first distractor arm structured and arranged to releasably engage a first bone screw;
a second distractor arm structured and arranged to releasably engage a second bone screw; and
a linkage mechanism connected to the actuator piston and the first and second distractor arms, wherein the linkage mechanism is structured and arranged to move the first and second distractor arms in relation to each other upon introduction of pressurized air into the pressurizable air cylinder,
and further comprising a controller structured and arranged to control the level of air pressure introduced into the air cylinder.
